(19) United States
(12) Patent Application Publication
(10) Pub. No.: US 2025/0228808 A1
(43) Pub. Date: Jul. 17, 2025

(54) COMPOSITIONS AND METHODS FOR THE TREATMENT OF CORONAVIRUS DISEASES

(71) Applicants: Laura LILE, Franklin, TN (US); Albert CRUM, Franklin, TN (US); PROTHIONE LLC, Franklin, TN (US)

(72) Inventors: Laura LILE, Franklin, TN (US); Albert CRUM, Franklin, TN (US)

(21) Appl. No.: 18/839,218

(22) PCT Filed: Feb. 15, 2023
(86) PCT No.: PCT/US2023/062653

§ 371 Date: Aug. 16, 2024

Related U.S. Application Data
(60) Provisional application No. 63/311,036, filed on Feb. 16, 2022.

(51) Int. Cl. A61K 31/198; A61K 31/122; A61P 31/14

(52) U.S. Cl. CPC A61K 31/198; A61K 31/122; A61P 31/14

(57) ABSTRACT

Compositions and methods for inhibiting coronavirus infection and for treating subjects already infected with a coronavirus such as the SARS-COV-2 virus that causes COVID-19. The compositions and methods are also useful for reducing the risk of developing severe CO VID-19 if a subject becomes infected with the SARS-COV-2 virus. The compositions and methods reduce viral load and reduce the time to reach clinical resolution in COVID-19 patients.

### FIELD OF THE INVENTION

The present disclosure generally relates to compositions and methods useful for reducing viral load, for mitigating symptoms of viral infection, and for reducing the likelihood of developing severe disease, in individuals infected with a coronavirus or at risk of becoming infected with a coronavirus (due to, e.g., likely exposure to infected individuals).

### BACKGROUND

As demonstrated by the recent COVID-19 pandemic, coronavirus infections can be fast-spreading and can cause severe illness and death in some individuals. Such mortality and serious illness, as well as the economic hardships endured during the pandemic, have highlighted the importance of having treatment options that are accessible, inexpensive, and broadly effective, and much effort has been devoted to developing new treatment options. See, e.g., Wu C. et al., Analysis of therapeutic targets for SARS-COV-2 and discovery of potential drugs by computational methods, Acta Pharamceutica Sinica B. 10 (5): 766-788 (2020).

### SUMMARY OF THE INVENTION

The compositions and methods of the present disclosure relate to coronavirus treatment options that are effective, inexpensive, and suitable for a broad range of patient groups, including pediatric patients.

Described herein are compositions and methods that are useful in treating subjects infected with a coronavirus, such as the SARS-COV-2 virus. The compositions and methods are also useful as a prophylaxis, to reduce the risk of becoming infected and/or of developing severe illness if infected with a coronavirus, such as the SARS-COV-2 virus. As demonstrated herein, administering a composition as described herein raises intracellular glutathione levels in individuals diagnosed with COVID-19, which is the disease caused by the SARS-CoV-2 virus. Administering a composition as described herein to COVID-19 patients also reduces viral load and the time required to reach clinical resolution, when compared to viral loads and times to clinical resolution observed in COVID-19 patients receiving placebo.

The compositions described herein generally comprise glycine, cystine, and a glutamate source; such components provide the amino acids required for glutathione synthesis. In certain embodiments, the compositions also comprise a selenium source and coenzyme Q10. Such compositions, when administered to subjects infected with a coronavirus or at risk of becoming infected with a coronavirus, efficiently deliver to cells components for glutathione synthesis, thereby raising intracellular glutathione levels and/or inhibiting the depletion of intracellular glutathione that can result from a coronavirus infection.

The present disclosure relates to these and other important aspects.

### BRIEF DESCRIPTION OF THE DRAWINGS

FIG. 1 is a Kaplan-Meier plot of time to clinical resolution (TTCR) by day 14 in Prothione™-treated patients versus placebo-treated patients (mITT population excluding days 0-2), as described in the Examples.

FIG. 2 is a bar plot showing the percentage of patients reaching clinical resolution by the specified time periods, i.e., days 0-5, days 6-10, days 11-15, days 16-20, days 21-25, and days 26-29 of the study described in the Examples.

FIG. 3 is a graph showing the time (in days) to clinical resolution for patients with unresolved COVID-19 at day 3 of the study described in the Examples.

### DETAILED DESCRIPTION OF THE INVENTION

The features and advantages of the present invention may be more readily understood by persons of ordinary skill in the art upon reading the following detailed description. It is to be appreciated that certain features of the invention that are described above and below in the context of separate embodiments may also be combined to form a single embodiment. Conversely, various features of the invention that are described in the context of a single embodiment for reasons of brevity may also be combined so as to form sub-combinations thereof. In addition, the drawings and specific embodiments of the invention described herein are illustrated by way of example, it being expressly understood that the description and drawings are only for the purpose of illustration and that the specific embodiments are not intended to define the limits of the present invention.

The present disclosure relates to methods of treating, and to methods of preventing severe symptoms of coronavirus diseases such as coronavirus disease 2019 (COVID-19), which is caused by the SARS-COV-2 virus. In general, the methods include administering to a subject a composition that increases the levels of intracellular glutathione. Such subject may be a person infected by the SARS-COV-2 virus or at risk of becoming infected with the SARS-COV-2 virus (e.g., a person living in a geographic area in which the SARS-COV-2 virus is endemic, or in which there is an outbreak of the virus). For example, the compositions described herein may be administered to individuals who have tested positive for COVID-19, who have been exposed to someone who has tested positive for COVID-19, and/or who are at high-risk of developing severe disease if infected with the virus.

Viral infections can deplete glutathione levels within cells, and cellular glutathione levels are decreased in cells infected with the SARS-COV-2 virus. Lee C., Therapeutic modulation of virus-induced oxidative stress via the Nrf2-dependent antioxidative pathway, Oxid Med Cell Longev. 2018:6208067; Ivanov A V et al., Oxidative Stress during HIV Infection: Mechanisms and Consequences. Oxid Med Cell Longev. 2016:8910396; Zhang Z et al., Flaviviridae Viruses and Oxidative Stress: Implications for Viral Pathogenesis, Oxid Med Cell Longev. 2019:1409582; Polonikov, A., Endogenous deficiency of glutathione as the most likely cause of serious manifestations and death in COVID-19 patients, ACS Infect Dis 6 (7): 1158-1562 (2020). Depleted levels of reduced glutathione (GSH) and oxidative stress may play a role in the pathogenesis of the viral infection and the development of severe COVID-19. Embodiments of the present invention relate to compositions and methods for raising intracellular GSH levels in virus-infected cells, by administering to COVID-19 patients components for GSH synthesis. Such raising of GSH levels is effective in mitigating COVID-19 symptoms and in reducing the duration and severity of the disease. In particular, as demonstrated herein, administering components for GSH synthesis to patients diagnosed with COVID-19 reduces patients' viral load, shortens the time for patients to reach clinical resolution, and may reduce the incidence of severe disease requiring hospitalization, when compared to patients administered a placebo. The present disclosure therefore relates to compositions and methods useful in the treatment of coronavirus diseases, including COVID-19 caused by the SARS-COV-2 virus, and to compositions and methods useful as a prophylaxis in individuals at risk of being infected and/or at risk of developing severe disease if infected.

Glutathione is a tripeptide of glycine, cysteine, and glutamic acid. Under normal physiological conditions, glutathione is present in cells in relatively high concentrations. See, e.g., van't Erve, Thomas J. et al., The concentration of glutathione in human erythrocytes is a heritable trait, Free Radic Biol Med., 65:742-749 (2013), which summarizes GSH levels reported in the literature and provides an estimated intracellular GSH concentration range of 0.4 to 3.0 mM (mean 1.4 mM). Glutathione maintains a reduced intracellular environment that protects the cell from oxidative stress. The thiol group in the cysteine of glutathione is a reducing agent and can be reversibly oxidized and reduced.

In addition to its protective role as an antioxidant, GSH also may block viral entry into cells and viral replication. Fraternale A. et al., GSH and analogs in antiviral therapy, Mol Aspects Med. 30:99-110 (2009). While not wishing to be bound by theory or mechanism: the cysteine residue of a glutathione molecule may be capable of inhibiting the replication of viruses that rely on Zn²⁺-binding proteins; the cysteine may serve as a binding site for the zinc in viral zinc finger proteins that are critical for the viral life cycle. By sequestering metals that the virus requires for replication and survival, the sulfhydryl of the cysteine in glutathione may protect a host cell from viral challenge.

Restoring virally-depleted GSH back to normal levels is challenging. For example, supplying GSH directly to cells is not a viable option, for several reasons. GSH synthesis is subject to negative feedback inhibition, such that supplying GSH to cells can halt native GSH synthesis and could lead to a dangerous rebound effect in patients. Ballatori N. et al., Glutathione dysregulation and the etiology and progression of human diseases, Biol Chem. 390 (3): 191-214 (2009). In addition, the half-life of GSH in the blood is on the order of only seconds to minutes. Lu S C, Regulation of glutathione synthesis, Curr Top Cell Regul. 36:95-116 (2000). Further, any GSH that remains in the blood must overcome thermodynamic and biochemical hurdles to enter the cell: for example, GSH is membrane-impermeable.

Instead of supplying GSH to cells, supplying cysteine, or a precursor of cysteine, N-acetyl cysteine (NAC), to cells has been explored. However, cysteine and NAC may compete with glutathione for resources in certain GSH recycling pathways, such that loading a cell with cysteine or with NAC may result in less efficient recycling of glutathione. In addition, NAC must be enzymatically de-acetylated before cysteine is made available for use in glutathione synthesis, and thus NAC is not able to increase intracellular GSH levels efficiently. Further, almost one-third of an NAC dose may be excreted by the kidneys. Sansone R A, Sansone L A, Getting a knack for NAC: N-Acetyl-Cysteine, Innov Clin Neurosci. 8 (1): 10-14 (2011).

In cases where increased GSH levels are desired, such as in patients infected with a coronavirus, a cystine-based product that includes the amino acid precursors for glutathione synthesis can be efficient at raising GSH levels, at least because there is no need for enzymatic de-acetylation (unlike for NAC).

Cystine can be rapidly converted to cysteine, which is the rate-limiting component of glutathione synthesis. Yildiz D. et al., Comparison of N-acetyl-L-cysteine and L-cysteine in respect to their transmembrane fluxes, Biochem Suppl Ser Membr Cell Biol. 3:157-162 (2009). Cystine is the natural extracellular reservoir of cysteine. Compared to cysteine, cystine has a longer half-life in the oxidized environment of the blood, and for at least some cell types it can readily move across the cell membrane. In addition, unlike for NAC, a cell's conversion of cystine to cysteine does not require an enzymatic reaction. Upon cellular entry, one molecule of cystine is reduced to two cysteines that are immediately available for GSH synthesis. The addition of selenium also may be beneficial to patients with GSH depletion, as selenium serves as a cofactor in GSH biosynthesis. Selenium deficiency also has been tied to COVID-19 outcomes. Moghaddam A. et al., Selenium deficiency is associated with mortality risk from COVID-19, Nutrients 12 (7): 2098 (2020).

Embodiments of the present invention generally relate to compositions comprising glycine, L-cystine, and a glutamate source. Such components provide the amino acids required for GSH synthesis. In certain embodiments, the glutamate source is glutamine and/or glutamic acid. In additional embodiments, the compositions further comprise a selenium source such as, for example, selenomethionine, selenocysteine, selenite, methylselenocysteine, and/or selenium nanoparticles. In certain embodiments, the amount of selenium source present in the composition, or the amount of selenium source that is administered to a subject, is sufficient to provide a dose of about 0.01 micrograms to about 20 micrograms of selenium. In further embodiments, the compositions also comprise coenzyme Q10.

In some embodiments, the compositions comprise glycine, L-cystine, a glutamate source, a selenium source, and coenzyme Q10. In certain such embodiments, the glutamate source is L-glutamine, and in further such embodiments, the selenium source is selenomethionine.

In any of the embodiments described herein, the glycine, cystine, and glutamate source (e.g., L-glutamine or L-glutamate) may be present in the composition as free-form amino acids. In addition, in any of the embodiments of the compositions and methods described herein, the stoichiometric ratio of glycine:cystine:glutamate administered to a subject can vary, e.g., from about 4:1:4 to about 1:4:1. In certain embodiments, the stoichiometric ratio is about 1:0.5:1.

In some embodiments, the present disclosure relates to a composition comprising glycine, L-cystine, a glutamate source (such as, e.g., L-glutamine or L-glutamic acid), and a selenium source (such as, e.g., selenomethionine, selenocysteine, or selenium particles), and optionally coenzyme Q10, for use in treating COVID-19, or for reducing the SARS-COV-2 viral load in a subject.

In certain embodiments, the composition comprises glycine, an L-glutamate source, L-cystine, and L-selenomethionine. In certain embodiments, the composition further comprises coenzyme Q10. In various embodiments, the composition further comprises a metallothionein or a fragment thereof.

In any of the various embodiments described herein, the composition may further comprise an additional agent that functions as a metal chelator. Such additional agent may be an Fe³⁺ chelator, a Zn²⁺ chelator, an Ni²⁺ chelator, or a combination thereof. The additional agent should be bio-compatible, and in some embodiments it may be desirable that the additional agent have a dissociation constant that is lower than the dissociation constant of relevant proteins (e.g., viral zinc finger proteins) that bind to the metal ions. Such agents may include, for example, zinc chelators such as N,N,N′,N′-tetrakis(2-pyridylmethyl)-ethylenediamine (TPEN), DPESA, TPESA, ethylenediaminetetraacetic acid (EDTA), ethylene glycol-bis(2-aminoethylether)-N,N,N′,N′-tetraacetic acid (EGTA), 1,2-bis(o-aminophenoxy) ethane-N,N,N′,N′-tetraacetic acid (BAPTA), and ethylenediamine-N,N′-diacetic-N,N′-di-β-propionic (EDPA), etc. and iron chelators such as diethylene triamine pentaacetic acid (DETAPAC), dipyridyl, pyridoxal isonicotinoyl hydrazone (PIH), desferrioxamine (DFO), deferiprone (DFP) and deferasirox (DFS).

In any of the various embodiments described herein, the composition may further comprise one or more of the following: an antiviral agent, an agent for treating fever, and a bronchodilator.

In certain embodiments, the present disclosure relates to use of a composition comprising glycine, L-cystine, a glutamate source selected from glutamine and glutamic acid, and a selenium source, optionally together with coenzyme Q10, for the manufacture of a medicament for treating COVID-19, or for reducing the SARS-COV-2 viral load in a subject. In some embodiments, the composition comprises a glycine, L-cystine, an L-glutamate source, L-selenomethionine, and coenzyme Q10.

A selenium source may comprise an inorganic selenium compound, e.g., an aliphatic metal salt containing selenium in the form of selenite or selenate anions, or an organic selenium compound, e.g., selenium cystine, selenium methionine, mono- or di-seleno carboxylic acids comprising about seven to eleven carbon atoms in the chain, or a seleno amino acid chelate. For a composition comprising glycine, L-cystine, and a glutamate source (e.g., glutamic acid or glutamine), the composition makes available two L-cysteines from the disulfide bond of L-cystine. When the composition also comprises L-selenomethionine, the composition further makes available an additional L-cysteine via transsulfuration of the methionine moiety in the selenomethionine. As L-cysteine is rate limiting for biosynthesis of glutathione, such compositions provide three L-cysteines, as well as the other amino acids needed for the synthesis of glutathione.

Example compositions include Immune Formulation 200™, which is a formulation of free-form amino acids and which comprises cystine, glycine, a glutamate source, and selenium. Immune Formulation 200 ™ has a favorable safety profile and is designed to overcome the hurdles discussed above for raising intracellular GSH levels. See generally US 2012/0029082; U.S. RE39,734; U.S. RE42,645; WO 2021/263206. In embodiments of methods as described herein, Immune Formulation 200™ can be administered with coenzyme Q10.

Another example composition is Prothione™. Prothione™ capsules comprise glycine, L-cystine, L-glutamine, selenomethionine, and coenzyme Q10. As described in the Examples herein, administering a Prothione™ composition to patients diagnosed with COVID-19 increases intracellular glutathione and reduces the duration and severity of the disease. When compared to COVID-19 patients taking placebo, COVID-19 patients taking Prothione™ showed a noticeable reduction in time to clinical resolution, which was defined as the time (in days) to attain three consecutive negative RT-PCR tests, each within 24-36 hours of the previous test. Patients treated with Prothione™ also showed a significant decrease in viral load of COVID-19, when compared to patients taking placebo.

Administering compositions comprising glycine, cystine, a glutamate source, and optionally further comprising a selenium source and coenzyme Q10 has several advantages over other anti-viral therapies. For example, the compositions described herein can be safely administered to subjects of all age groups, as the dose of the composition can be selected such that the amount of selenium (if present in the composition) administered is below the selenium upper intake limit for infants and children (as set by the Food and Nutrition Board at the Institute of Medicine of the National Academies). There is also little concern for drug interactions.

Embodiments of the invention relate to methods of administering, to a subject diagnosed with COVID-19 or to a subject exposed to COVID-19, for example, the following: 1177.5 mg glycine, 600.3 mg L-cystine, 1175.1 mg L-glutamine, 6 mg coenzyme Q10, and 0.017 mg selenomethionine. In certain such embodiments (such as for pediatric patients), the 1177.5 mg glycine, 600.3 mg L-cystine, 1177.5 mg L-glutamine, 6 mg coenzyme Q10, and 0.017 mg selenomethionine are administered once daily, and in other embodiments (such as for adult patients), the 1177.5 mg glycine, 600.3 mg L-cystine, 1177.5 mg L-glutamine, 6 mg coenzyme Q10, and 0.017 mg selenomethionine are administered twice daily (e.g., in the morning and in the evening). It should be understood that the amounts described herein are approximations and encompass a range within limits typically accepted in the pharmaceutical industry.

In further embodiments, a patient diagnosed with COVID-19, or an individual exposed to COVID-19 or suspected of having COVID-19, is administered Prothione™. In some embodiments, Prothione™ is administered as three 1-gram Prothione™ capsules, either once daily (for a total daily dose of 3 grams Prothione™) or twice daily (for a total daily dose of 6 grams Prothione™). For certain embodiments involving pediatric subjects under 3 years of age and/or under 40 kg in weight, three 1-gram Prothione™ capsules (or other dosage form such as a solution or suspension comprising the same amounts of glycine, L-glutamine, L-cystine, coenzyme Q10, and selenomethionine as are present in three 1-gram Prothione™ capsules) are administered once daily. In alternative embodiments involving pediatric subjects (e.g., patients at least 3 years of age and under 40 kg in weight), a pediatric subject is administered 588 mg glycine, 588 mg L-glutamine, 300 mg L-cystine, 3 mg coenzyme Q10, and 0.0085 mg selenomethionine twice daily (e.g., in the morning and in the evening).

In some embodiments, the methods described herein further comprise administering to a subject a metal chelator, in addition to the glycine, cystine, glutamate source, and in some embodiments also the selenium source and coenzyme Q10. The metal chelator may be an Fe³⁺ chelator, a Zn²⁺ chelator, an Ni²⁺ chelator, or a combination thereof. The metal chelator may be included in any of the compositions described herein, or may be administered separately.

In some embodiments, the methods comprising administering to a subject glycine, cystine, a glutamate source, and optionally a selenium source and coenzyme Q10 as described herein, further comprise administering to the subject a metallothionein or fragment thereof.

In certain embodiments, the methods described herein (such as a method of administering a composition as described herein to a subject) increase the subject's intracellular levels of reduced glutathione (GSH). In some embodiments, the methods described herein elevate the subject's intracellular concentration of glutathione by at least about 25%, at least about 30%, at least about 40%, at least about 50%, at least about 60%, at least about 70%, at least about 80%, at least about 90%, at least about 100%, at least about 120%, at least about 150%, or at least about 200%, compared to the subject's pre-treatment level of intracellular glutathione. In various embodiments, administering a composition as described herein to a subject elevates the subject's intracellular concentration of glutathione by at least about 40%. In various embodiments, administering a composition as described herein to a subject allows the subject to reach an intracellular concentration of glutathione that is from 0.1 mM to 4.0 mM, by 24-48 hours following the first administration of the composition. For example, in certain embodiments an intracellular concentration of glutathione that is from 0.5 mM to 3.0 mM is reached by 24 hours after the first administration. In various embodiments, the administration of the composition to a subject is effective in allowing the subject to reach an intracellular concentration of glutathione that is from 2 mM to 4 mM, by 72 hours post-first administration. In some embodiments, administering a composition as described herein to a subject results in an intracellular concentration of glutathione that is at least 0.3 mM, at least 0.4 mM, at least 0.5 mM, at least 0.6 mM, at least 0.7 mM, at least 0.8 mM, at least 0.9 mM, at least 1.0 mM, at least 1.5 mM, at least 2.0 mM, at least 2.5 mM, at least 3.0 mM, at least 3.5 mM, or that is 4.0 mM, by 72 hours, or by 48 hours, following the first administration of the composition.

In certain embodiments, a composition as described herein is administered at a dose and frequency that is effective to reduce or inhibit depletion of intracellular glutathione levels in coronavirus-infected cells at 24-72 hours following the first administration of the composition. In some embodiments, administering a composition as described herein is effective to restore intracellular glutathione levels in SARS-COV-2 virus-infected cells to the intracellular glutathione levels in non-infected cells at 24-48 hours following the first administration of the composition. A first administration of the composition may be before or after infection with the SARS-COV-2 virus, or before or after exposure to someone infected with the virus. In certain embodiments, the composition is first administered after infection with the SARS-COV-2 virus, and in some embodiments is first administered after an exposure to someone infected with the virus. In specific embodiments, the composition is first administered from about 12 hours to about 96 hours post-infection with the SARS-COV-2 virus (or similarly may be administered from about 12 hours to about 96 hours post-exposure). For example, the first administration of the composition to a subject may be from about 24 hours to about 72 hours post-infection with the SARS-COV-2 virus (or similarly, from about 24 hours to about 72 hours post-exposure). In various embodiments, the composition is first administered about 48 hours post-infection with the SARS-COV-2 virus (or similarly, about 48 hours post-exposure).

As described above, a “first” administration need not be the first time a subject has ever been administered the composition; rather, first administration refers to the first dose or first administration in a given series of administrations (e.g., for a treatment schedule involving twice-daily administration for ten days, the first administration would be the earlier (e.g., morning) administration occurring on day 1 of those ten days, even if the subject had been administered the composition a week prior to day 1).

In certain embodiments, administering a composition as described herein to a subject having COVID-19 reduces the subject's viral load by at least about 10%, at least about 15%, at least about 20%, at least about 25%, at least about 30%, at least about 40%, at least about 50%, at least about 60%, at least about 70%, at least about 80%, at least about 90%, or at least about 95%, compared to the subject's viral load at baseline. In some embodiments, administering a composition as described herein to a subject having COVID-19 reduces the subject's viral load by at least about 50% or more. In specific embodiments, the composition comprises glycine, L-cystine, L-glutamine, selenomethionine, and coenzyme Q10, each present in about the same proportional amount as found in Prothione™ capsules, and wherein the glycine, L-cystine, and L-glutamine are present as free-form amino acids.

In some embodiments, administering a composition as described herein to a subject diagnosed with COVID-19 reduces the time required for the subject to reach clinical resolution (indicated herein by 3 negative RT-PCR tests as described herein), compared to the average time to reach clinical resolution observed for untreated or placebo-treated COVID-19 subjects. For example, in certain embodiments, subjects testing positive for COVID-19 are administered a composition as described herein (e.g., with twice-daily administration) beginning on day 0 and reach clinical resolution by day 4, by day 5, by day 6, by day 7, by day 8, by day 9, by day 10, by day 11, by day 12, by day 13, by day 14, or by day 15 of treatment.

Embodiments of the present invention also relate to methods for post-exposure prophylaxis of COVID-19, especially for patients who would be at high risk of progression to severe COVID-19 requiring hospitalization, if infected. Compositions and methods described herein are also useful for individuals who are not fully vaccinated or who are not expected to mount an adequate immune response to complete SARS-COV-2 vaccination (for example, individuals with immunocompromising conditions, including patients taking immunosuppressive medications), and/or for individuals who have been exposed to an individual infected with SARS-COV-2, and/or for individuals who are at high risk of exposure to an individual infected with SARS-COV-2 (for example, because of occurrence of SARS-COV-2 infection in other individuals in the same institutional setting, such as nursing homes, prisons, etc.). Compositions as described herein can be administered as soon as possible after an exposure to someone with COVID-19, or as soon as possible after receiving a positive RT-PCR test result for the virus.

Individuals at high risk for progression to severe COVID-19 include individuals having one or more of the following medical conditions or other factors: older age (for example, age >65 years of age), younger age (for example, <1 year old), obesity or being overweight, pregnancy, chronic kidney disease, diabetes, immunosuppressive disease or receiving immunosuppressive treatment, cardiovascular disease (including congenital heart disease), hypertension, chronic lung diseases (for example, chronic obstructive pulmonary disease, asthma (moderate-to-severe), interstitial lung disease, cystic fibrosis, and pulmonary hypertension), sickle cell disease, neurodevelopmental disorders (for example, cerebral palsy), other conditions that confer medical complexity (for example, genetic or metabolic syndromes and severe congenital anomalies), and having a medical-related technological dependence (for example, tracheostomy, gastrostomy, or positive pressure ventilation (not related to COVID-19)).

Certain embodiments of the present disclosure relate to prophylactic methods of reducing the ability of a coronavirus to infect cells, and of reducing the development of severe disease if a subject becomes infected, comprising administering a composition as described herein. For example, in certain embodiments, a subject having one or more of the conditions or factors described above is administered a composition as described herein (e.g., three 1-gram Prothione™ capsules once-daily, or three 1-gram Prothione™ capsules twice daily), during a coronavirus pandemic or local outbreak, for example, as a pre-cautionary measure to reduce the ability of the virus to infect cells and replicate if the subject is exposed to the virus, and/or to mitigate severe disease if the subject becomes infected.

### Examples

A randomized, double-blinded, placebo-controlled clinical trial was conducted to evaluate the safety and efficacy of Prothione™ capsules in the treatment of patients with mild to moderate COVID-19.

Study subjects were adults ≥18 years of age diagnosed with COVID-19 by a standardized RT-PCR assay and having mild to moderate symptoms associated with COVID-19. Patients were deemed to have mild disease if they suffered mild symptoms such as fever, rhinorrhea, mild cough, sore throat, malaise, headache, and/or muscle pain, and did not suffer from shortness of breath or exhibit signs of a more serious lower airway disease (respiratory rate <20 breaths/minute, heart rate <90 beats/minute, and oxygen saturation (pulse oximetry)>93% on room air). Moderate disease included the symptoms above as well as more significant lower respiratory symptoms, such as shortness of breath (at rest or with exertion), and signs of moderate pneumonia but without signs of more serious lower airway disease (respiratory rate >20 but <30 breaths/minute, heart rate ≥90 but less than 125 beats/minute, oxygen saturation (pulse oximetry)>93% on room air, and if available, lung infiltrates based on X-ray or CT scan <50% present). Key exclusion criteria were: severe COVID-19 disease; signs of acute respiratory distress syndrome (ARDS) or respiratory failure necessitating mechanical ventilation at the time of screening; and history of systemic corticosteroids.

A total of 231 COVID-19 patients enrolled in the study and were randomized to the treatment arm (115 patients) or placebo arm (116 patients). Approximately half of patients in each arm had four or more COVID-19 symptoms (e.g., fever, dyspnea, cough, and myalgia) at the time of enrollment. During the trial, seven subjects withdrew from the treatment arm and seventeen subjects withdrew from the placebo arm. Due to withdrawals, protocol deviations, and loss to follow-up, 107 patients in the treatment arm and 97 patients in the placebo arm completed the study.

Subjects in the treatment arm were administered three 1-gram Prothione™ capsules (3 grams Prothione™) orally twice a day, for thirty days, for a total daily dose of 6 grams Prothione™ Each Prothione™ capsule contains glycine, L-glutamine, L-cystine, coenzyme Q10, and selenomethionine. Six grams of Prothione™ daily provides a daily dosage of: glycine (2355 mg), L-glutamine (2355 mg), L-cystine (1200.6 mg), coenzyme Q10 (12 mg), and selenomethionine (0.034 mg). Subjects in the placebo arm were administered a placebo twice daily.

Within three days of the initial screening visit, study subjects began the 30-day treatment period. Following the treatment period, subjects had two additional visits for follow-up, at 7 days and 30 days after the last dose.

Clinical endpoints included the following:
Time (in days) to clinical resolution (TTCR); clinical resolution was reached when a subject had three consecutive negative RT-PCR tests, each conducted within 24-36 hours of the previous test. TTCR was the primary outcome measured.
Clinical improvement in fever, dyspnea, cough, and/or myalgia, as determined by, e.g., Clinical Symptom Score Assessment (CCSA) (score 0-12).
Progression of disease and hospitalizations.
Change from baseline in red blood cell intracellular glutathione levels.
COVID-19 viral load, measured on days 0, 1, 2, 3, 4, 5, 6, 7, 10, 14, 21, and 29 or until a subject had three consecutive negative RT-PCR tests as described above.
   Incidence of treatment-related adverse events (TRAEs), and incidence and severity of treatment-emergent adverse events (TEAEs) were monitored, as were changes in blood chemistry, ECG, and vitals. This trial involved 14,091 clinical visits with study subjects.

Subjects treated with Prothione™ capsules showed a mean change in COVID-19 viral load, from baseline to day 14, that was significantly greater compared to the mean change in COVID-19 viral load in the placebo arm (see Table 1). Additionally, there was an increase in red blood cell GSH levels from baseline to day 29 in Prothione™-treated patients compared to placebo-treated patients, with a mean intracellular GSH of 156.9 μM in the treatment arm (n=95) versus 107.3 μM (n=83) in the placebo arm. In addition, for subjects with moderate disease, the placebo arm had a larger number of such subjects with a decrease in GSH levels from baseline to day 29 (13 out of 17 (76.5%)), compared to the Prothione™-treatment arm (with 4 out of 17 (23.5%) having a decrease in GSH levels, from baseline to day 29). Further, for subjects with moderate disease and showing an increase in GSH levels from baseline to day 29, there were 12 out of 19 (64%) of such subjects in the Prothione™-treatment arm, compared to 7 out of 19 (36%) of such subjects in the placebo arm.

During the trial, there was a lower number of hospitalizations in Prothione™-treated patients, with only one hospitalization in the Prothione™-treatment arm (n=103) compared to four COVID-19 related hospitalizations in the placebo arm (n=101). In addition, a total of 25 days of hospitalization occurred during the trial, with 5.9 (23.6%) of these days for the subject in the Prothione™-treatment arm, compared to 19.1 (76.4%) of these days for subjects in the placebo arm. These results demonstrates that the decrease in viral load helped reduce progression and severity of disease.

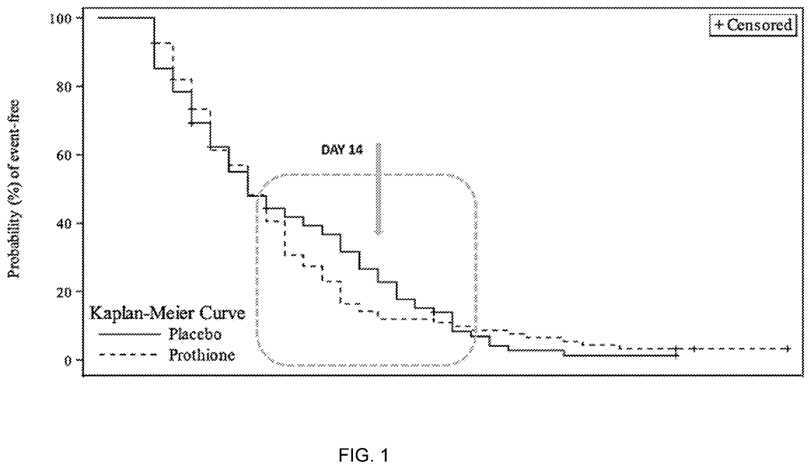

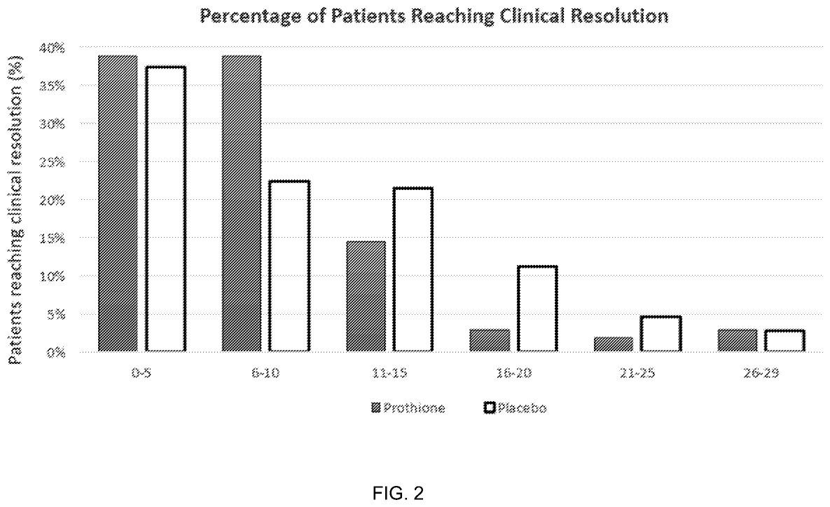

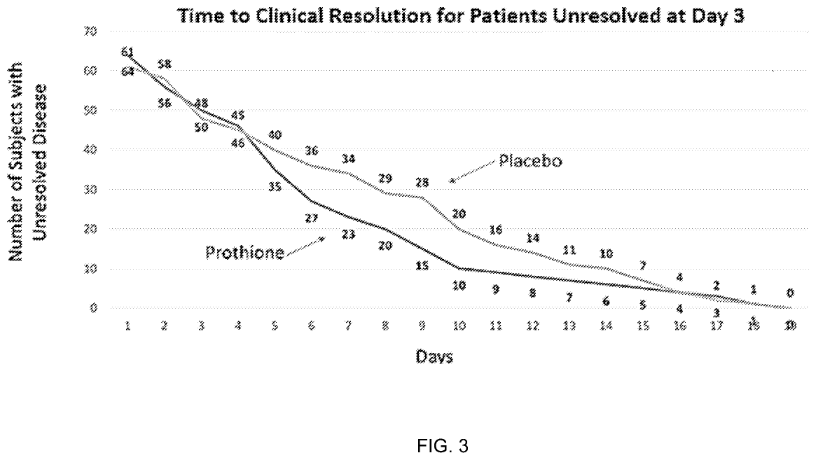

Patients in the Prothione™-treatment arm also recovered more quickly compared to patients in the placebo arm. The time to clinical resolution (TTCR) was shorter in the treatment arm compared to placebo, as shown in FIGS. 1-3. While many patients with COVID-19 recovered by day 14, as shown in FIG. 2, 77.7% of patients in the Prothione™-treatment arm reached clinical resolution by day 10, whereas 59.8% of placebo-treated patients reached clinical resolution by day 10. In addition, as shown in FIG. 3, for patients with unresolved disease at day 3, Prothione™ administration promoted faster resolution. Furthermore, patients with moderate COVID-19 appeared to have a greater response to Prothione™ administration due to lower GSH levels at the time of entering the trial.

This study further showed that Prothione™ capsules are safe, with no difference in safety measures compared to placebo. There were a total of 25 Treatment Related Adverse Events (TRAEs). No TRAEs led to discontinuations or withdrawals from the study. In addition, there was no clustering of events for a single system, and many TRAEs were related to incidental diagnoses. No renal signals or abnormal liver enzymes were observed, and no changes in lymphocytes or platelets, lipid metabolism, glucose levels, or electrolytes were observed.

TABLE 1
Summary of Mean Change from Baseline to Day
14 in Viral Load (c/mL) (mITT population)

| | | Prothione ™ (n = 103) | | Placebo (n = 101) | | |
|---|---|---|---|---|---|---|
| | | | Change from | | Change from | |
| Timepoint | Statistics | Result | baseline | Result | baseline | p-value |
| Baseline | n | 100 | | 94 | | |
| | Mean | 27400000 | | 24600000 | | |
| | SD | 95100000 | | 85400000 | | |
| | Median | 174966 | | 87014 | | |
| | Min | 0 | | 0 | | |
| | Max | 739000000 | | 616000000 | | |
| Day 14 | n | 22 | 22 | 39 | 39 | |
| | Mean | 43337 | −64500000 | 7039.7 | −22400000 | 0.0347 |
| | SD | 119470 | 177000000 | 39214 | 75200000 | |
| | Median | 0 | −249000 | 0 | −99538 | |
| | Min | 0 | −739000000 | 0 | −416000000 | |
| | Max | 525188 | 427529 | 245075 | 5361.4 | |

TABLE 2
Summary of Other Key Outcomes

| | Number of subjects | Number of subjects with clinical |
|---|---|---|
| | hospitalized | resolution before day 14 |
| Prothione ™ | 1 | 85 (82.5%) |
| Placebo | 4 | 71 (70.3%) |

While this invention has been particularly shown and described with references to certain embodiments thereof, it will be understood in light of the present disclosure by those skilled in the art that various changes in form and details may be made therein without departing from the scope of the invention, for example as encompassed by the appended claims.

1. A method for reducing viral load in a subject infected with a coronavirus, the method comprising administering to the subject, on a daily basis and for at least three consecutive days, either one of the following:
about 2,355 mg glycine, about 2,355 mg L-glutamine, about 1,200 mg L-cystine, about 12 mg coenzyme Q10, and about 0.034 mg selenomethionine; or
about 1,177 mg glycine, about 1,177 mg L-glutamine, about 600 mg L-cystine, about 6 mg coenzyme Q10, and about 0.017 mg selenomethionine.

2. The method according to claim 1, wherein the about 2,355 mg glycine, about 2,355 mg L-glutamine, about 1,200 mg L-cystine, about 12 mg coenzyme Q10, and about 0.034 mg selenomethionine are administered, and wherein the subject is an adult human.

3. The method according to claim 1, wherein the subject is administered the about 2,355 mg glycine, about 2,355 mg L-glutamine, about 1,200 mg L-cystine, about 12 mg coenzyme Q10, and about 0.034 mg selenomethionine, or the about 1,177 mg glycine, about 1,177 mg L-glutamine, about 600 mg L-cystine, about 6 mg coenzyme Q10, and about 0.017 mg selenomethionine, for at least five consecutive days.

4. (canceled)

5. The method according to claim 1, wherein the about 1,177 mg glycine, about 1,177 mg L-glutamine, about 600 mg L-cystine, about 6 mg coenzyme Q10, and about 0.017 mg selenomethionine are administered, and wherein the subject is a human of at least 3 years of age and weighing no more than about 40 kg.

6. (canceled)

7. The method according to claim 1, wherein a composition comprises:
the about 2,355 mg glycine, about 2,355 mg L-glutamine, about 1,200 mg L-cystine, about 12 mg coenzyme Q10, and about 0.034 mg selenomethionine; or
the about 1,177 mg glycine, about 1,177 mg L-glutamine, about 600 mg L-cystine, about 6 mg coenzyme Q10, and about 0.017 mg selenomethionine; and
wherein the administering comprises orally administering to the subject the composition once-daily or twice-daily for at least three consecutive days.

8. (canceled)

9. The method according to claim 7, wherein the composition comprises the about 1,177 mg glycine, about 1,177 mg L-glutamine, about 600 mg L-cystine, about 6 mg coenzyme Q10, and about 0.017 mg selenomethionine, and wherein the subject is a human of at least 3 years of age and weighing no more than about 40 kg.

10. A method of preventing a coronavirus infection in a subject, the method comprising orally administering to the subject a composition comprising either one of the following:
about 2,355 mg glycine, about 2,355 mg L-glutamine, about 1,200 mg L-cystine, about 12 mg coenzyme Q10, and about 0.034 mg selenomethionine; or
about 1,177 mg glycine, about 1,177 mg 1a-glutamine, about 600 mg L-cystine, about 6 mg coenzyme Q10, and about 0.017 mg selenomethionine.

11. The method according to claim 1, wherein the coronavirus is SARS-COV-2 virus.

12. The method according to claim 10, wherein the coronavirus infection is a SARS-COV-2 infection.

13. Use of glycine, L-glutamine, L-cystine, coenzyme Q10, and selenomethionine in the preparation of a medicament for treating SARS-COV-2 infection in a subject.

14. (canceled)

15. The method according to claim 7, wherein the composition comprises the about 2,355 mg glycine, about 2,355 mg L-glutamine, about 1,200 mg L-cystine, about 12 mg coenzyme Q10, and about 0.034 mg selenomethionine, and wherein the subject is an adult human.

16. The method according to claim 7, wherein the composition is administered for at least five consecutive days.

17. The method according to claim 10, wherein the composition comprises the about 2,355 mg glycine, about 2,355 mg L-glutamine, about 1,200 mg L-cystine, about 12 mg coenzyme Q10, and about 0.034 mg selenomethionine, and wherein the subject is an adult human.

18. The method according to claim 10, wherein the composition comprises the about 1,177 mg glycine, about 1,177 mg L-glutamine, about 600 mg L-cystine, about 6 mg coenzyme Q10, and about 0.017 mg selenomethionine, and wherein the subject is a human of at least 3 years of age and weighing no more than about 40 kg.

19. The method according to claim 10, wherein the composition is administered to the subject once-daily or twice daily, for at least three consecutive days.

20. The method according to claim 19, wherein the composition is administered for at least five consecutive days.
